(19) United States
(12) Patent Application Publication
(10) Pub. No.: US 2007/0244420 A1
(43) Pub. Date: Oct. 18, 2007

(54) Ankle brace

(76) Inventor: Barry Paul Boden, Chevy Chase, MD (US)

Correspondence Address: RR Law Firm;(Attn: Paul L. Ratcliffe), 42870 Meander Crossing Ct., Ashburn, VA, 20148, US

(21) Appl. No.: 11/603,414

(22) Filed: Feb. 12, 2007

Related U.S. Application Data
(60) Provisional application No. 60/739,331, filed on Nov. 23, 2005.

(51) Int. Cl. A61F 5/00

(52) U.S. Cl. 602/27

(57) ABSTRACT

An ankle brace for restricting dorsiflexion and promoting plantarflexion of the foot relative to the leg. The ankle brace includes a lower leg assembly worn on the wearer lower leg, a foot assembly worn on the wearer's foot; and one or more hinge assemblies rotatably connecting the lower leg assembly to the foot assembly. The hinge assembly provides a positive or resistive force on the top foot assembly to prevent dorsiflexion and/or to promote plantarflexion to reduce knee injuries.

This application claims the benefit or Unites States Provisional Patent Application 60/739,331 filed on Nov. 23, 2005, the entirety of which is incorporated herein by reference.

This application includes material which is subject to copyright protection. The copyright owner has no objection to the facsimile reproduction by anyone of the patent disclosure, as it appears in the Patent and Trademark Office files or records, but otherwise reserves all copyright rights whatsoever.

### FIELD OF THE INVENTION

The present invention relates generally to orthopedic braces and, in particular, to ankle braces, foot braces, ankle supports, or foot supports.

### BACKGROUND OF THE INVENTION

The human ankle and knee joints involve complex mechanisms providing for extension, flexion, and rotary movement. The bones, cartilage, and ligaments provide a myriad of components within the joints making them susceptible to injury. Known ankle braces are focused on supporting the components within the ankle itself with no thought or design provided to prevent or assist with injuries to other joints including the knee.

One of the most severe injuries to the knee is an anterior cruciate ligament (“ACL”) rupture. The ACL is also one of the most commonly disrupted ligaments in the knee. It is estimated that each year in the United States there are Epidemiologic studies have documented a significantly higher ACL injury rate in women as compared to male athletes, especially in team sports that require cutting and pivoting such as basketball, football, and soccer. Women suffer two to eight times the number of ACL injuries in basketball and soccer as men.

Research into ACL injuries, including videotape analysis of athletes who sustained an ACL injury, provided that landing on a flat foot with the knee close to full extension and often in valgus (outward turning of the lower leg in relation to the thigh) predisposes one to an ACL injury. Therefore, ACL injury prevention programs emphasize teaching athletes to land on the balls of their feet with the knees flexed avoiding valgus. Test results of these prevention programs demonstrate a reduction in the incidence of ACL injuries.

Further, since the leg acts as a kinetic chain with the forces starting at the foot and traveling up to the knee, the ankle is a logical location for an ACL prevention device. If deleterious ground reaction forces are not dissipated at the lower leg, it is unlikely that a knee brace can effectively reduce these forces below the threshold to prevent ACL injury.

Therefore, what is needed is an ankle brace or support which provides an appropriate amount of resistance to the foot or ankle joint to properly position the foot for landing on the forefoot (balls of their feet) thereby preventing ACL injuries.

### SUMMARY OF INVENTION

Accordingly, the present invention is directed to an orthopedic device or brace for the ankle or foot.

An object of the present invention is to promote the proper position of the ankle or foot when landing on the foot to prevent knee injuries including ligament injuries.

The present invention provides an ankle brace or foot support device that provides resistance to dorsiflexion (flexion). The brace has several advantages over a knee brace in terms of preventing an ACL injury including: (1) If the ankle is properly positioned in plantarflexion (extension) at landing, a reflex automatically places the knee in flexion often avoiding the vulnerable knee position; (2) Patients are usually more compliant in wearing ankle braces since they are less cumbersome than large bulky knee braces; and (3) The ankle brace could be designed to have the dual purpose of providing medial and lateral ankle support to reduce the incidence of ankle sprains. For clarification, dorsiflexion is movement of the foot or toes upward toward the front of the shin and plantarflexion is movement of the foot or toes downward away from the front of the shin.

The present invention provides an ankle brace comprising: (1) a lower leg assembly adapted to be worn on a user's lower leg; (2) a foot assembly adapted to be worn on the user's foot; and (3) at least one hinge rotatably connecting said lower leg assembly to said foot assembly, wherein said at least one hinge provides a force on said foot assembly to prevent dorsiflexion and/or to promote plantarflexion. The lower leg assembly may be comprised of a flexible sleeve and/or a pair of brace panels and at least one strap or any combination thereof. The foot assembly may be comprised of a flexible sleeve, a plastic molded form with or without padding, or panels and straps.

To achieve these and other advantages and in accordance with the purpose of the present invention, as embodied and broadly described, in one aspect of the present invention there is provided an ankle or foot brace for providing a resistance to the foot to properly position the foot for landing on the forefoot thereby preventing knee injuries.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings. It is to be understood that both the foregoing general description and the following detailed description are exemplary and explanatory and are intended to provide further explanation of the invention as claimed.

### BRIEF DESCRIPTION OF DRAWINGS

The accompanying drawings, which are included to provide a further understanding of the invention, are incorporated herein, and constitute a part of this specification and illustrate embodiments of the invention. The figures with the description serve to explain the principles of the invention.

FIG. 1 is a front elevation view of an ankle brace embodying the present invention positioned on a user's ankle.

FIG. 2 is a side elevation view of the ankle brace shown in FIG. 1.

FIG. 3 is an isometric view of portions of the ankle brace shown in FIG. 1.

FIG. 4 is a partial view of the ankle brace shown in FIG. 1.

FIG. 5 is a side elevation view of an ankle.

### DETAILED DESCRIPTION

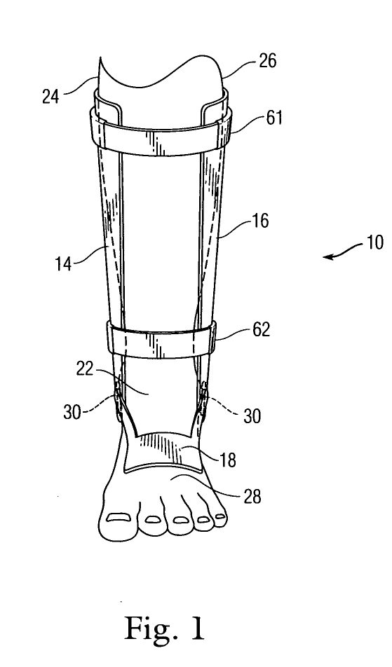

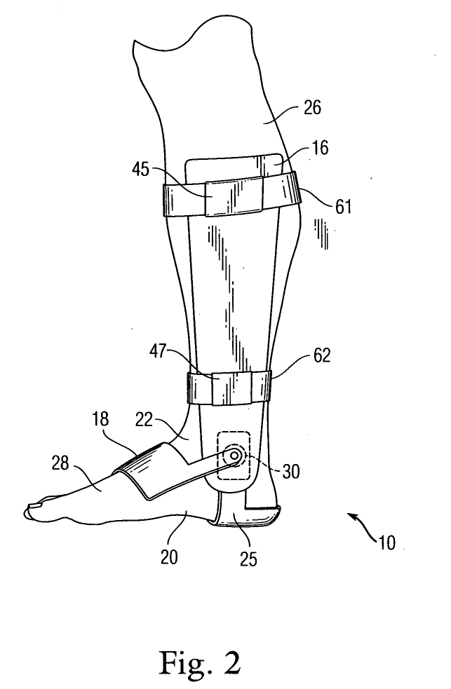

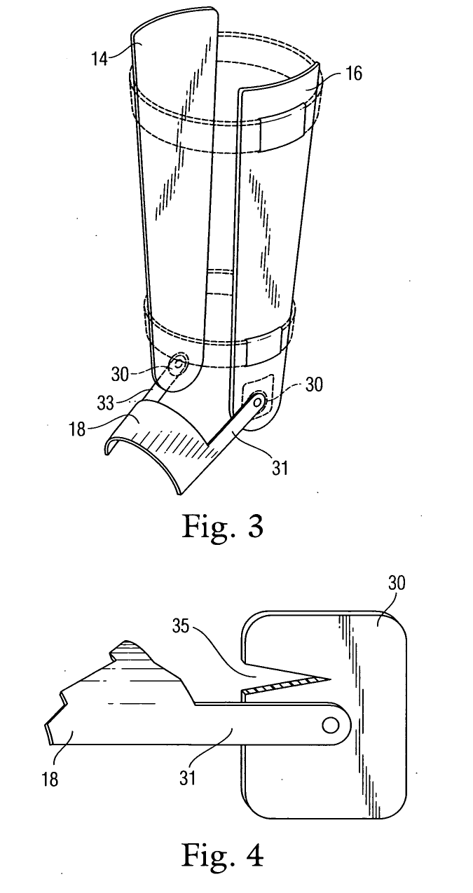

The system of the present invention will now be described in conjunction with FIGS. 1-5.

The present invention provides a new ankle brace 10 or support which provides a gradually increasing resistance to ankle dorsiflexion to decrease the likelihood of knee injuries. As previously indicated, research has shown that knee injuries to the ACL are often tracked back to improper landing on a flat foot or on the heel with the knee close to extension and in valgus. The present invention provides an ankle brace 10 which provides an appropriate resistive force to the foot 20 or ankle joint 22 to properly position the foot 20 to land on the forefoot thereby preventing common knee injuries.

As seen in FIGS. 1-5, the brace 10 includes a standard pair of panels 14, 16 on the medial 24 and lateral 26 aspects of the ankle 22. In addition, the brace 10 contains a dorsum panel 18 that covers the dorsum 28 (top) of the foot 20. In the preferred embodiment, the dorsum panel 18 is made from molded plastic and includes padding to protect the wearer's foot 20. The preferred embodiment also includes side panels 14, 16 which are connected to the dorsum panel 18 through one or more hinges 30 located on one or both of the side panels 14, 16. The side panels 14, 16 may be cast or molded plastic to fit the wearer's inside ankle or leg portion 24 and outside ankle and leg portion 26. The hinge 30, at the bottom portion of side panels 14, 16, is connected to the dorsum panel 18 by arms, support straps, or rods 31, 33. The dorsum panel 18 provides a resistance to the top of the foot 28 which helps the user to resist dorsiflexion and/or promote plantarflexion so the wearer is more likely to land on the forefoot. The brace 10 may also employ a heel pad or support 25 connected to the side panels 14, 16 to provide more rigidity to the ankle brace.

The hinge 30 may be a mechanical hinge such as a spring biased hinge or frictional teeth and gear hinge, an air or gas pressured hinge, or other known hinge designs capable of providing the necessary resistance or force. An example of a mechanical hinge assembly which might be employed is provided in U.S. Pat. No. 6,527,733 which is incorporated herein by reference in its entirety. Further, as seen in FIG. 4, the hinge 30 may provide a void 35 and/or mechanical wedge which enables the user to vary the degree or amount of resistance to dorsiflexion.

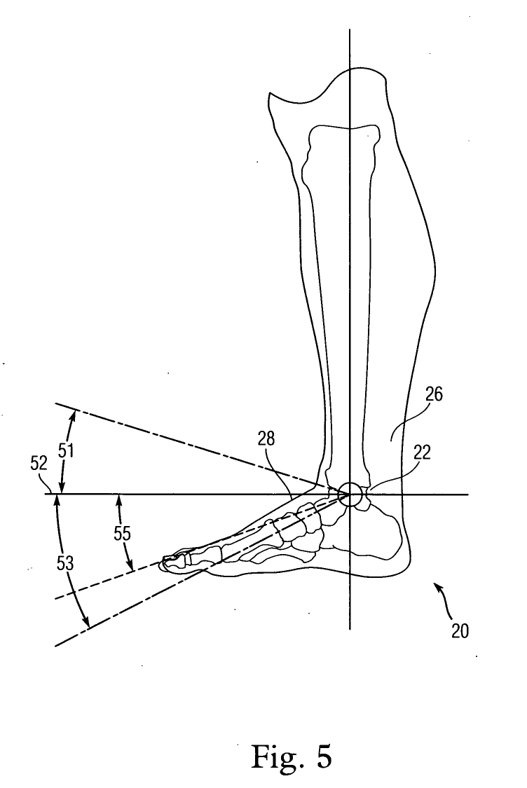

The mean ankle dorsiflexion and plantarflexion in males age 20 to 59 is 26.1+/−6.5 and 40.5+−8.1 respectively (Nigg). In females age 20 to 59 the mean dorsiflexion and plantarflexion is 24.9+−6.1 and 44.4+/−7.7 respectively (Nigg). As seen in FIG. 5, the foot can dorsiflex in an upward angle 51 above the horizontal plane 52 or plantarflex in a downward angle 53 below the horizontal plane 52. The ankle or foot brace 10 of the present invention may provide a gradually increasing resistance to ankle dorsiflexion as the foot moves from a plantarflexed to a dorsiflexed position. With usage and test data the resistance levels at various degrees of dorsiflexion and plantarflexion can be determined for a user providing variances in the resistance levels based upon the angle of the foot to optimize the brace 10 to prevent common injuries to the knee. In the preferred embodiment, the ankle brace 10 will not provide resistance until the user has moved the ankle past some pre-set range of motion or angle 55 (FIG. 5) below the horizontal plane 52, such as a set angle within the range of minus 5 to minus 25 degrees dorsiflexion (or +5 to 25 degrees plantarflexion). This would allow the user some movement and flexibility of the ankle 22 without strong resistance but promote proper foot placement.

The dorsum panel 18 may be made of plastic, fabric, leather, or any other appropriate material or substance. The dorsum panel 18 may be adjustable or of a material that can be form fitted for the user of the brace 10 such as neoprene, injection molded plastic, or other similar materials. Further, the dorsum panel 18 may be larger, smaller, or of a different shape than that specifically depicted in the figures. Further, the dorsum panel 18 may be adjustable in shape and size or easily removed from the brace 10 to allow the wearer to attach different size and shape dorsum panels 18. The brace 10 may be formed as a one or multi-pieced sleeve or sock such as a neoprene sleeve. In one embodiment, the side panels 14, 16 are fitted and secured to the user's ankle 22 using adjustable straps 61, 62. These adjustable straps 61, 62 may employ known adjustment features such as the use of hook and loop or Velcro type elements to adjust and secure the straps 61, 62 to the proper size for the user's ankle 22. Still further, the side panels 14, 16 may be custom fitted using molded plastic such that the panels 14, 16 and straps 61, 62 can be properly fitted for each individuals ankle 22. Further, the side panels 14, 16 could be formed with molded strap protrusion sections 45, 47 which allow the straps 61, 62 to be threaded through the side panels 14, 16 while maintaining a form fit.

In addition, the present invention can employ the use of sensors would be located on the bottom of the brace such as at the sole of the foot. The sensor or sensors would detect the removal or a reduction in pressure. The sensor upon sensing a pressure reduction would notify and therefore activate the hinge 30 to begin increasing resistance or to apply a force to dorsum panel 18. The sensors would enhance the brace 10 such that no force is applied to the dorsum panel 18 to promote plantarflexion when the user's foot 20 is on the ground but would activate the hinge 30 when the user's foot 20 is in motion and off of the ground. Still further, the sensor could include a time delay such that the hinge 30 is only activated after some amount of time has lapsed after the pressure on the foot 20 has been reduced or removed. For example, if the average time between normal walking steps is 1 second but the average time between athletic running and jumping steps is 1.4 seconds the ankle brace 30 could employ a delay (i.e. 1 second) between when the sensor senses a reduction or removal in pressure and the hinge 30 provides a resistance or applies a force to the user's foot.

The brace 10 could also employ learning technology which measures and learns the user's use profile such that it measures the user's typical walking gate or time between steps and calculates or adds a pre-determined time delay on top of the measured walking time to determine the appropriate delay before the hinge 30 provides a resistance or begins to apply force. Still further, the sensors need not be on the brace 10 and could be employed on or in the user's shoe, sock, or some neoprene type sleeve where the sensor is in communication with the brace 10 and/or hinge 30.

While the invention has been described in detail and with reference to specific embodiments thereof, it will be apparent to those skilled in the art that various changes and modifications can be made therein without departing from the spirit and scope thereof.

What is claimed is:

1. An ankle brace comprising:
a lower leg assembly adapted to be worn on a user's lower leg;
a foot assembly adapted to be worn on said user's foot;
at least one hinge rotatably connecting said lower leg assembly to said foot assembly;
wherein said at least one hinge provides a force on said foot assembly to prevent dorsiflexion.

2. The ankle brace of claim 1, wherein said lower leg assembly is comprised of a flexible sleeve.

3. The ankle brace of claim 1, wherein said lower leg assembly is comprised of a pair of brace panels and at least one strap.

4. The ankle brace of claim 1, further comprising a heel assembly attached to said lower leg assembly which is adapted to be worn on said user's heel.

5. An ankle brace comprising:
a lower leg assembly adapted to be worn on a user's lower leg;
a foot assembly adapted to be worn on said user's foot;
at least one hinge rotatably connecting said lower leg assembly to said foot assembly;
wherein said at least one hinge provides a force on said foot assembly to promote plantarflexion.
